(19) United States
(12) Patent Application Publication
(10) Pub. No.: US 2003/0120340 A1
(43) Pub. Date: Jun. 26, 2003

(54) Mitral and tricuspid valve repair

(76) Inventors: Jan Liska, Stockholm (SE); Paul Liska, Stockholm (SE); Peter Liska, Saltsjobaden (SE)

Correspondence Address: YOUNG & THOMPSON, 745 SOUTH 23RD STREET 2ND FLOOR, ARLINGTON, VA, 22202

(21) Appl. No.: 10/025,472

(22) Filed: Dec. 26, 2001

(51) Int. Cl. A61F 2/24

(52) U.S. Cl. 623/2.1; 623/2.36; 623/2.11

(57) ABSTRACT

The present invention relates to the correction of mitral and tricuspid valve regurgitation. More particularly this means that he leaflet bases of the posterior and anterior mitral leaflets are connected to each other with a stabilizing element (14) extended transversely across the valve annulus (13). Said stabilizing element (14) is by first (22) and second (24) ends secured into a first (26) and a second (28) position respectively at said valve annulus (13) by means of sutures, anchors or clips. Coaptation is attained, by reducing the length of the stabilizing element (14) from D1 to D2.

### TECHNICAL FIELD OF THE INVENTION

The invention relates to the correction of mitral and tricuspid valve regurgitation. More particularly, the invention relates to methods and means according to the preamble of the independent claims, for a simplified and less invasive repair of a mitral or tricuspid heart valve with significant regurgitation.

### BACKGROUND OF THE INVENTION

The mitral valve is comprised of an anterior leaflet and a posterior leaflet. The bases of the leaflets are fixed to a circumferencial partly fibrous structure, the annulus, preventing dehiscence of the valve. A subvalvular apparatus of chordae and papillary muscles prevents the valve from prolapsing into the left atrium. Mitral valve disease can be expressed as a complex variety of pathological lesions of either valve or subvalvular structures, but can also be related to the functional status of the valve. Functionally the mitral valve disease can be categorized into two anomalies, increased leaflet motion i.e. leaflet prolaps leading to regurgitation, or diminished leaflet motion i.e. restricted leaflet motion leading to obstruction and/or regurgitation of blood flow.

Leaflet prolaps is defined as when the free edge of the leaflet overrides the plane of the orifice during ventricular contraction. The mitral regurgitation can also develop secondary to alteration in the annular ventricular apparatus and altered ventricular geometry, followed by incomplete leaflet coaptation. In ischemic heart failure this can be attributed to papillary or lateral wall muscle dysfunction, and in non-ischemic heart failure it can be ascribed to annular dilation and chordal tethering, all as a result of dysfunctional remodeling.

The predominant cause to dysfunction of the mitral valve is regurgitation which produces an ineffective cardiac pump function resulting in several deleterious conditions such as ventricular and atrial enlargement, pulmonary hypertension and heart-failure and ultimately death.

The main objective for the surgical correction is to restore normal function and not necessarily anatomical correction. This is accomplished by replacing the valve or by reconstructing the valve. Both of the procedures require the use of cardiopulmonary bypass and is a major surgical operation carrying a non-negligible early morbidity and mortality risk, and a postoperative rehabilitation for months with substantial postoperative pain. Historically, the surgical approach to patients with functional mitral regurgitation was mitral valve replacement, however with certain adverse consequences such as thromboembolic complications, the need for anticoagulation, insufficient durability of the valve, loss of ventricular function and geometry.

Reconstruction of the mitral valve is therefore the preferred treatment for the correction of mitral valve regurgitation and typically consists of a quadrangular resection of the posterior valve (valvuloplasty) in combination with a reduction of the mitral valve annulus (annuloplasty) by the means of suturing a ring onto the annulus. These procedures are surgically demanding and require a bloodless and well-exposed operating field for an optimal surgical result. The technique has virtually not been changed for more than three decades.

Recently a new technique has been adopted for repairing prolaps of the valve by anchoring the free edge of the prolapsing leaflet to the corresponding free edge of the opposing leaflet and thereby restoring apposition but not necessarily coaptation. Therefore a ring annuloplasty is also required to attain complete coaptation.

This method commonly referred to as an edge-to-edge repair also has certain drawbacks such as the creation of a double orifice valve and thereby reducing the effective orifice area. Several less invasive approaches related to the edge-to-edge technique has been suggested, for repairing mitral valve regurgitation by placing a clip through a catheter to suture the valve edges. However, it still remains to conduct an annuloplasty procedure, which has not yet been resolved by a catheter technique and therefore is to be performed by conventional surgery, which makes the method impractical.

When repairing the mitral valve by means of cardiopulmonary bypass and cardiac arrest with the valve visually exposed, the correct length and size of the device is assessed as follows. One or several polypropylene mattressed stay sutures are extended transversely across the valves and attached to the anterior leaflet base and the posterior leaflet base respectively, which stay-sutures are then snared and tourniquet. The length of each stay-suture can thus be shortened and adjusted until the valves become competent when testing the valve competence by means of filling the left ventricle with saline under pressure. When the valve is competent the distance between the transverse suture points is measured, which distance is to correspond to the length of the stabilizing element being selected. Then, the propylene stay sutures are removed and the stabilizing element is attached and secured to the respective valve leaflet base and deep into the annulus with a suture or clip means at the corresponding points as of the previously used stay sutures.

Advantageously, a less invasive approach to the left atrium is possible, commonly referred to as the transeptal catheterization technique. This conventional technique is well known from the literature and used for different purposes such as pressure measurements in the left atrium or radiofrequency ablation in the left atrium or intervention with a balloon to dilate a stenotthrombocytopeniastitchic mitral valve. By inserting a transeptal sheath device percutaneuosly into the femoral vein and advance it through the inferior vena cava into the right atrium and subsequently puncture through the intra-atrial septum with a Brockenbrough needle at the level of the fossa ovalis, the left atrium is accessed. Thereafter the trocar and dilator of the device is removed, leaving the sheath in position in the left atrium.

### OBJECT OF THE INVENTION

The present invention aims to solve problems associated with achieving easily reproducible, rational and durable methods and means for repairing mitral valve regurgitation, which does not require complex procedures such as annuloplasty or valve reconstruction and involves the possibility of a less invasive approach. In particular it is desirable that said repairing be performed on a beating heart such that the patient does not have to be placed on cardiopulmonary bypass.

### SUMMARY OF THE INVENTION

According to the present invention the solution is achieved by the methods according to the characterizing features of independent claims 1-4 and by means of the characterizing features of independent claims 11-14. In principle this means that the leaflet bases of the posterior and anterior mitral leaflets are connected to each other with a stabilizing element extended transversely across the valve at one or multiple points. Advantageous improvements and developments of the invention appear from the dependent claims.

### DRAWING SUMMARY

The invention will be described in more detail in the following description, with reference to the accompanying schematic drawing.

FIG. 1 discloses a mitral valve having a dilated annulus (bad coaptation),

FIG. 2 is a cross section of the mitral valve in FIG. 1,

FIG. 3 discloses said mitral valve being repaired by means of stabilizing elements (coaptation attained),

FIG. 4A is a cross section of the repaired mitral valve in FIG. 3,

FIG. 4B is an upscaled sectional view of a stabilizing element embodied by a rod or wire,

FIG. 5 discloses a mitral valve with a mitral prolaps (bad apposition),

FIG. 6 is a cross section of the mitral valve in FIG. 5,

FIG. 7 discloses said mitral valve provided with a stabilizing element for repairing said mitral prolaps (apposition attained),

FIG. 8 is a cross section of the repaired mitral valve in FIG. 7,

FIGS. 9 and 10 disclose advantageous embodiments of a stabilizing element for a sectional mitral prolaps and,

FIGS. 11-18 are step-by-step-views, which disclose one variant of a means for the endovascular repair of a dilated annulus.

### DETAILED DESCRIPTION OF THE INVENTION

As previously mentioned above, a cardiac valve as shown in FIG. 1, particularly a mitral valve 2, is comprised of an anterior leaflet 4 and a posterior leaflet 6, each with a base 8 and 10 and an edge 11 and 12 respectively. Said bases are fixed to a circumferencial partly fibrous structure, the annulus 13, preventing dehiscence of the valve. For clarity reasons, said leaflets 4; 6 have been divided into three sections A, B and C, which will be described in more detail later.

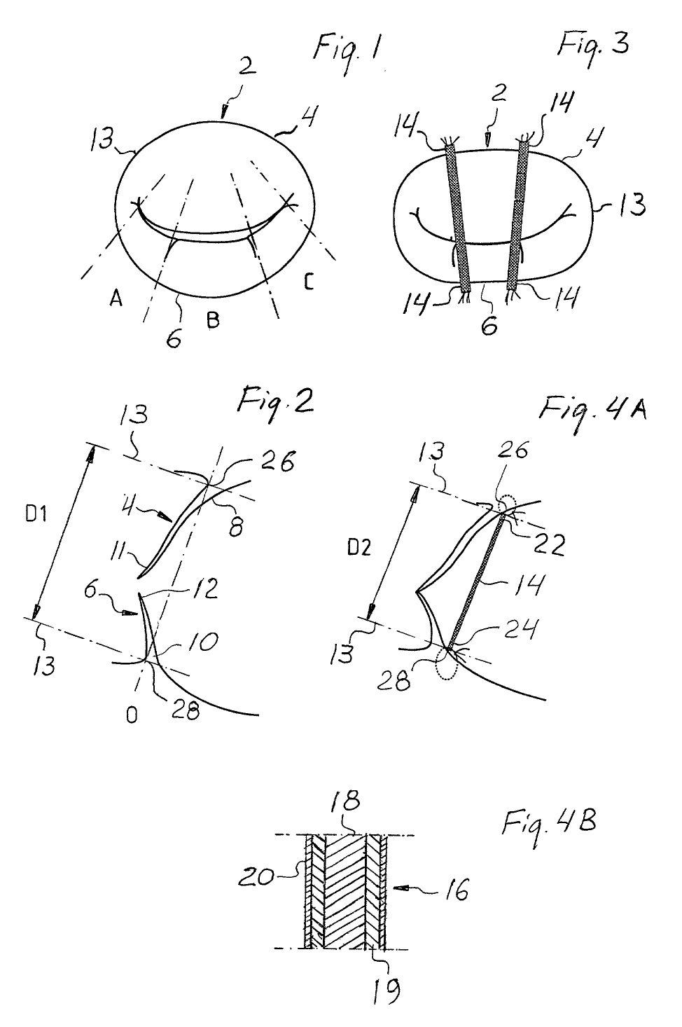

In FIGS. 1 and 2 the mitral valve 2 is disclosed in a condition where annular dilation or tethering of the chordae is present. According to the invention coaptation of the leaflets might be obtained by reducing the distance between the anterior 8 and posterior 10 leaflet bases respectively, by means of one or more stabilizing elements 14 (FIGS. 3-4).

According to FIG. 4B, each stabilizing element 14 might be designed as at least one rod or wire 16 with a core 18 of metal to obtain a specific stiffness. Said core 18 might be embedded in a plastic material or covered by a polyester fabric, to obtain a bio-compatible cover 19. Said cover might be provided with a surface coating 20 of a smooth plastic material e.g. polytetrafluoroethylene (PTFE). Additionally, said cover 19 makes it possible to attach and secure said stabilizing element 14 (rod or wire 16) to each leaflet base 8; 10 in a conventional manner with a surgical suture, a surgical clip etc., as for example is shown in FIG. 4A. Further, each stabilizing element 14 might be shaped of one or more rods or wires 16 or the like as disclosed in the drawing (strip, band, net-shaped etc.) for a multiple point fixation. It might also consist of a tread or band made of polytetrafluoroethylene (PTFE) or nitinol, which have excellent durable and bio-compatible properties. Independent of its shape, each stabilizing element 14 has a first 22 and second 24 end, each to be attached to the respective leaflet base 8; 10.

As appear from FIGS. 1-4A, the dilated valve 2 has a first position 26 at the anterior leaflet base 8 and a second position 28 at the posterior leaflet base 10, which are located at a mutual distance D1. By means of two band-shaped stabilizing elements 14 of suitable lengths, which with the respective first ends 22 are secured to the respective first position 26 and with the respective second ends 24 are secured to the respective second position 28, said distance D1 can be reduced to a distance D2, whereby coaptation is attained and the valve is made competent again.

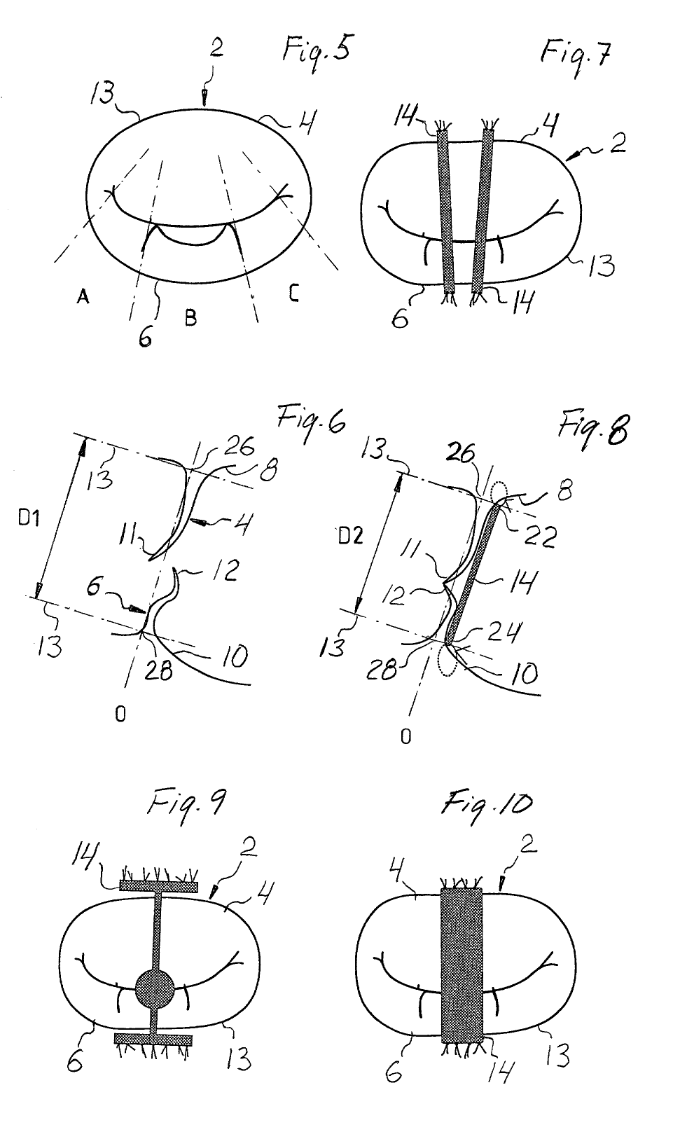

In the case of leaflet prolaps of a specific leaflet segment, as shown in FIGS. 5-10 segment B, said stabilizing element or elements 14 might be arranged between the two leaflet bases 8 and 10 respectively, at the atrial side of the prolaps. The stabilizing element 14 thereby mechanically restricts the free edge 12 of the prolapsing leaflet segment B to override the plane of orifice O. Further, this arrangement will also result in apposition of the leaflets, at the same time coaptation is attained by reduction of the distance D1 to D2 between the anterior 8 and posterior 10 leaflet bases.

Advantageously, the stabilizing element or elements 14 might be introduced into the left atrium and secured to the different positions in there by means of the above mentioned transeptal catheterization technique.

According to one embodiment of the invention, as shown in FIGS. 11-18, an interventional catheter 30 of conventional design with a tip 31 at its distal end is advanced into the left atrium through a not shown sheath of a conventional kind. Said sheath might be preformed and/or steerable to orient said tip 31 of said interventional catheter 30 inside the left atrium and relative to the mitral leaflet bases 8; 10. The catheter orientation might be monitored by the use of fluoroscopy and/or echocardiography. By the intervention of the catheter, a first applicator 32 at the catheter tip 31 might be positioned at the posterior mitral leaflet base 10.

In FIGS. 11-12, a stabilizing element in the form of a doubled thread or a band 14, advantageously made of PTFE or Nitinol, is attached by means of said first applicator 32 and, by means of a spiral shaped first anchor or clip 34 made of Nitinol, anchored into the fibrous part of the leaflet base 10 at the annulus 13. The first anchor or clip 34 is put in place by the first applicator 32 and actuated via the catheter 30 by means of a conventional, not shown, release mechanism controlled from the proximal end of the catheter 30.

Firstly, to achieve the anchoring, said anchor or clip 34 is preferably rotated to an optional extent by means of the catheter 30. Due to its spiral shape, the rotation will drive the first anchor or clip 34 to a definable depth into the annulus 13. Alternatively, the first anchor or clip 34 might be anchored into the annulus 13 by means of a pincher movement. Secondly, the PTFE or Nitinol threads or band is fixed to the first anchor or clip 34 and is extruded from the tip 31 at said distal catheter end by means of feeding the threads or band through the catheter from the proximal catheter end at a desirable length. This enables that the band or threads 14 are not limiting further maneuverability of the catheter tip 31 at the distal end of the catheter 30.

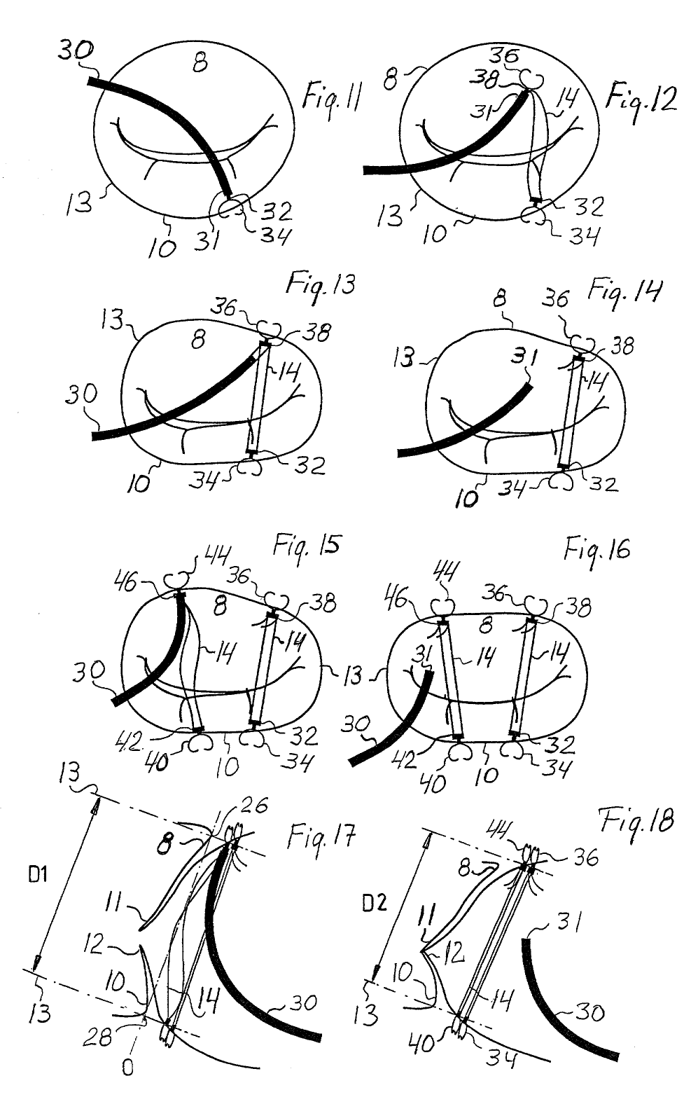

As disclosed in FIGS. 12-14, the catheter tip 31 is then repositioned transversely across the valve orifice to the anterior mitral valve leaflet base 8. A second anchor or clip 36 is attached and released from a second applicator 38 into the fibrous part of the valve base 8 and anchored into the annulus 13 in a similar way as the first anchor or clip 34. The band or threads 14 can freely move through the second applicator 38 and through the second anchor or clip 36. By means of retracting the band or threads 14 through the catheter by pulling the threads or band 14 at the proximal end of the catheter 30, the threads or band 14 is stretched and the distance D1 between the first 34 and second 36 anchored clips or anchors can be reduced to the distance D2 (FIG. 13).

By the use of transesophageal echocardiography, the function of the mitral valve can be assessed and when the valve 2 is competent on the relevant section (1/2 B and C), the threads or band 14 is fixed to the second anchor or clip 36 located at the anterior leaflet base 8. This fixation is employed by a not shown, third applicator deploying a likewise not shown fixation clip of a conventional design, from the distal catheter end 31 and releasing it by the not shown release mechanism located at the proximal catheter end. Said fixation can also be made by ultrasonic welding technique. Finally, the threads or band 14 is cut just proximal to the respective anchor, by means of a not shown internal cutter located just proximal to the distal catheter end 31. Even the cutter can be released (not shown) from the proximal catheter end (FIG. 14).

The different interventional tools, (first applicator 32, first clip 34, second applicator 38, second clip 36, fixation clip etc.) can either be all contained in the catheter 30 or be exchanged for each step of the procedure. This completes the measures related to one of the stabilizing elements 14. In case more than one stabilizing element 14 is used or a single stabilizing element with multiple fixation points, the above-described steps are carried out repeatedly. Consequently, as in this case two stabilizing elements are used, the second stabilizing element 14 is attached to the annulus 13 with similar steps and corresponding interventional tools as the first one.

Therefore, a third anchor or clip 40 is put in place at the posterior leaflet base 10 by a third applicator 42 and anchored into the annulus 13 by means of the catheter 30. Then, the catheter tip 31 is repositioned again transversely across the valve orifice to the anterior leaflet base 8. At this position a fourth anchor or clip 44 is put in place by a fourth applicator 46 and anchored into the annulus 13 by means of the catheter 30. Also the second band or threads 14 can freely move through the fourth applicator 46 and second anchor or clip 44.

Likewise, by means of retracting the second band or threads 14 through the catheter by pulling the threads or band 14 at the proximal end of the catheter 30, the threads or band 14 is stretched and also the distance D1 between the third 40 and fourth 44 anchored clips or anchors can be reduced to the distance D2 (FIGS. 15-18). Again by the use of transesophageal echocardiography, the function of the mitral valve can be assessed. When the valve 2 is entirely competent, that is even on the remaining section (A and 1/2 B), the second threads or band 14 is fixed to the fourth anchor or clip 44 located at the anterior leaflet base 8. The same steps regarding fixation are carried out as mentioned before (FIGS. 14, 16-18).

After completion of all the steps of the procedure the catheter 30 and not shown guidance sheath are retracted from the left atrium and extracted from the venous access port.

The number of stabilizing elements 14 to be fixed to the leaflet bases 8; 10, their design and exact orientation, depend on the underlying causes to the mitral valve regurgitation. For example when annular dilation and/or tethering of chordae are the pathophysiological etiology to the valve dysfunction, typically one or two (or more) stabilizing elements 14 are placed proportionally over the valve as shown in FIGS. 1-4. When a prolaps of a segment is the cause of valve regurgitation, typically two bands or a pair of rods are arranged over the prolapsing segment as shown in FIGS. 5-10. Alternatively one or two stabilizing elements, for example bands 14, are placed over the lesion as shown in FIGS. 7, 9 and 10, in dependence of the specific characteristics of said prolaps. Even the stabilizing elements in the form of two doubled threads 14, used in the embodiment shown in FIGS. 11-18, might be used as an individual doubled thread placed symmetrically over a lesion, e.g. in the central part of segment B.

Approximation of the anterior and posterior mitral valve bases with a stabilizing element extended transversely across the valve orifice is a new and previously not described technique for repairing an incompetent mitral valve. Said technique hereafter referred to as the base-to-base repair.

According to the described embodiments a simple and effective repair technique is provided relative to the complex and surgically demanding approaches of conventional methods such as chordal shortening, valve resection, chordal transposition, artificial chordae replacement or ring annuloplasty.

Even if the edge-to-edge mitral valve repair is a relatively new and simple technique, it is ineffective without concomitant ring annuloplasty, thereby making the procedure more complex and therefore less attractive. In the less invasive intravascular approach for applying the base-to-base technique it is not necessary to grasp the valve leaflets. This fact makes it an easier procedure to perform on a beating heart as compared to an instrumental edge-to-edge procedure, where the heart frequency most likely has to be reduced substantially.

The base-to-base repair can be advantageously combined with other cardiac surgery procedures such as coronary artery bypass grafting minimizing the ischemic damage for the cardioplegic arrested heart by reducing the ischemic time. The base-to-base repair also provides an approach of a less invasive procedure without the trauma of open-heart surgery and cardiopulmonary bypass. Thus, the procedure can be accomplished concomitant with percutaneous transluminal coronary angioplasty (PTCA) or as a stand-alone outpatient procedure in a cardiac catheterization laboratory. The advantages include reduced cost, hospitalization and patient recovery times. With minimal trauma to the patient, it may be desirable to perform the repair earlier before the disease has progressed to a serious level. Thus, more repair procedures may be performed, preventing further progression of the disease, obviating the need for more serious invasive procedures.

Consequently, according to the present invention advantageous means have been developed for mitral valve repair with preferred embodiments described in details herein. This description is an exemplification only of the principles of the invention and is not intended to limit the invention to the particular embodiments described.

1. A method for the repair of a cardiac valve (2) provided with an anterior (4) and posterior (6) leaflet, each being protruding from a corresponding leaflet base (8; 10) at the valve annulus (13), comprising the following steps: modifying said cardiac valve (2) by bringing at least one stabilizing element (14) into a selected position (26; 28) at each of the leaflet bases (8, 10); interconnecting said leaflet bases by extending said stabilizing element (14) across the cardiac valve (2) and; determining the distance (D1, D2) between the anterior (8) and posterior (10) leaflet bases by means of said stabilizing element (14).

2. A method for the repair of a cardiac valve (2) provided with an anterior (4) and posterior (6) leaflet, each being protruding from a corresponding leaflet base (8; 10) at the valve annulus (13), comprising: entering a stearable applicator (32) endovascularly into the left atrium; modifying said cardiac valve (2) by using said stearable applicator (32) for performing the following steps: bringing at least one stabilizing element (14) into a selected position (26; 28) at each of the leaflet bases (8, 10); interconnecting said leaflet bases by extending said stabilizing element (14) across the cardiac valve (2) and; determining the distance (D1, D2) between the anterior (8) and posterior (10) leaflet bases by means of said stabilizing element (14).

3. A method for the repair of a cardiac valve (2) provided with an anterior (4) and posterior (6) leaflet, each being protruding from a corresponding leaflet base (8; 10) at the valve annulus (13), comprising the following steps: modifying said cardiac valve (2) by bringing a first end (22) of at least one stabilizing element (14) into a first selected position (26) of the valve annulus (13) and a second end (24) of said at least one stabilizing element (14) into a second selected position (28) of the valve annulus (13); interconnecting said leaflet bases (8; 10) by extending said stabilizing element (14) across the cardiac valve (2) and; determining the distance (D1, D2) between the anterior (8) and posterior (10) leaflet bases by means of said stabilizing element (14).

4. A method for the repair of a cardiac valve (2) provided with an anterior (4) and posterior (6) leaflet, each being protruding from a corresponding leaflet base (8; 10) at the valve annulus (13), comprising: entering a stearable applicator (32) endovascularly into the left atrium; modifying said cardiac valve (2) by using said stearable applicator (32) for performing the following steps: bringing a first end (22) of at least one stabilizing element (14) into a first selected position (26) of the valve annulus (13) and a second end (24) of said at least one stabilizing element (14) into a second selected position (28) of the valve annulus (13); interconnecting said leaflet bases (8; 10) by extending said stabilizing element (14) across the cardiac valve (2) and; determining the distance (D1, D2) between the anterior (8) and posterior (10) leaflet bases by means of said stabilizing element (14).

5. A method according to one of claims 1-4, comprising the step of: adjusting the distance (D1, D2) between the anterior (8) and posterior (10) leaflet bases by varying the length of said stabilizing element (14).

6. A method according to one of claims 1-5, comprising the step of: attaching the stabilizing element (14) to the atrial side of each leaflet base (8; 10), said stabilizing element (14) serving as a support for said leaflets (4, 6).

7. A method according to claim 6, comprising the steps of: tightening the stabilizing element (14), in case of a prolaps, by means of shortening its length between its selected positions (26; 28) at said leaflet bases (8; 10) close to the apposition line (O) of the leaflets (4, 6) (the orifice plane of the valve), thereby extending said stabilizing element 14 straightly between said selected positions (26; 28); locating even a central part of said stabilizing element 14 between said leaflet bases (8; 10) close to the apposition line (O) of the leaflets (4, 6) and; at the same time positioning the leaflet bases (8; 10) mutually closer and attaining coaptation of the leaflets (4, 6).

8. A method according to one of claims 2-7, comprising the steps of: encasing said stearable applicator (32) (catheter) in an inserting device (guidance sheath) for penetrating the human skin and achieving a venous access port; extending the stearable applicator (32) from a maneuvering device at a proximal end outside said access port, through the femoral vein, the inferior vena cava and the right atrium to penetrate the intra-atrial septum to the left atrium and; arranging the stearable applicator (32) (catheter) with a manipulative distal end (31) in one of said selected positions (26; 28).

9. A method according to claim 8, which is comprised of assessing said selected position (28) to the posterior mitral leaflet base (10).

10. A method according to claim 8, which is comprised of assessing said selected position (26) to the anterior mitral leaflet base (8).

11. A means for the repair of a cardiac valve (2) comprising an anterior (4) and posterior (6) leaflet, each being protruding from a corresponding leaflet base (8; 10) at the valve annulus (13), said means being arranged for modifying said cardiac valve (2), in which the distance (D1, D2) between the anterior (8) and posterior (10) leaflet bases is determined by means of at least one stabilizing element (14), which has been brought into a selected position (26; 28) at each of the leaflet bases (8, 10), said leaflet bases being interconnected by means of said stabilizing element (14) which is extended across the cardiac valve (2).

12. A means for the repair of a cardiac valve (2) comprising an anterior (4) and posterior (6) leaflet, each being protruding from a corresponding leaflet base (8; 10) at the valve annulus (13), said means being arranged for modifying said cardiac valve (2), in which the distance (D1, D2) between the anterior (8) and posterior (10) leaflet bases is determined by means of at least one stabilizing element (14), which by means of a stearable applicator (32) for endovascular entrance into the left atrium, has been brought into a selected position (26; 28) at each of the leaflet bases (8, 10), said leaflet bases being interconnected by means of said stabilizing element (14) which is extended across the cardiac valve (2).

13. A means for the repair of a cardiac valve (2) comprising an anterior (4) and posterior (6) leaflet, each being protruding from a corresponding leaflet base (8; 10) at the valve annulus (13), said means being arranged for modifying said cardiac valve (2), in which the distance (D1, D2) between the anterior (8) and posterior (10) leaflet bases is determined by means of at least one stabilizing element (14), which is provided with a first (22) and a second (24) end, which has been brought into a first (26) and a second (28) selected position of the valve annulus (13), said leaflet bases (8; 10) thereby being interconnected by means of said stabilizing element (14), which is arranged to be extended across the cardiac valve (2).

14. A means for the repair of a cardiac valve (2) comprising an anterior (4) and posterior (6) leaflet, each being protruding from a corresponding leaflet base (8; 10) at the valve annulus (13), said means being arranged for modifying said cardiac valve (2), in which the distance (D1, D2) between the anterior (8) and posterior (10) leaflet bases is determined by means of at least one stabilizing element (14), which is provided with a first (22) and a second (24) end, which, by means of a stearable applicator (32) for endovascular entrance into the left atrium, has been brought into a first (26) and a second (28) selected position of the valve annulus (13), said leaflet bases (8; 10) thereby being interconnected by means of said stabilizing element (14), which is arranged to be extended across the cardiac valve (2).

15. A means according to one of claims 11-14, wherein the distance (D1, D2) between the anterior (8) and posterior (10) leaflet bases is adjustable by means of varying the length of said stabilizing element (14).

16. A means according to claim 15, wherein the stabilizing element (14) is attached to the atrial side of each leaflet base (8; 10) and serves as a support for said leaflets (4, 6).

17. A means according to claim 16, wherein the stabilizing element (14), in case of a prolaps, by means of shortening its length, is tightened between its selected positions (26; 28) at said leaflet bases (8; 10) close to the apposition line (O) of the leaflets (4, 6) (the orifice plane of the valve), said stabilizing element 14 thereby being straightly extended between said selected positions (26; 28), whereby even a central part of said stabilizing element 14 between said leaflet bases (8; 10) is located close to the apposition line (O) of the leaflets (4, 6), at the same time as the leaflet bases (8; 10) are mutually closer positioned and coaptation of the leaflets (4, 6) attained.

18. A means according to one of claims 11-17, wherein the stabilizing element (14) is comprised of a rod or wire.

19. A means according to one of claims 11-17, wherein the stabilizing element (14) is comprised of a number of rods or wires.

20. A means according to one of claims 11-17, wherein the stabilizing element (14) is a structure comprising a number of rods or wires.

21. A means according to one of claims 11-17, wherein the stabilizing element (14) is comprised of a strip or band.

22. A means according to one of claims 11-17, wherein the stabilizing element (14) is comprised of a number of strips or bands.

23. A means according to claim 21 or 22, wherein each strip or band is net-formed.

24. A means according to one of claims 20-22, wherein an intermediate section of each structure, strip or band is shaped in the form of a ring.

25. A means according to one of claims 20-22, wherein an intermediate section of each structure, strip or band is shaped in the form of a circular disc.

26. A means according to one of claims 11-25, wherein said stearable applicator (32) (catheter) is encased in an inserting device (guidance sheath) for penetrating the human skin to achieve a venous access port and extended, from a maneuvering device at a proximal end outside said access port, through the femoral vein, the inferior vena cava and the right atrium to penetrate the intra-atrial septum to the left atrium, the stearable applicator (32) (catheter) being arrangable with a manipulative distal end (31) in one of said selected positions (26; 28).

27. A means according to claim 26, wherein the selected position (28) is assessed to the posterior mitral leaflet base (10).

28. A means according to claim 26, wherein the selected position (26) is assessed to the anterior mitral leaflet base (8).
